(19) United States
(12) Patent Application Publication
(10) Pub. No.: US 2016/0220143 A1
(43) Pub. Date: Aug. 4, 2016

(54) BODY COMPOSITION MEASURING APPARATUS AND METHOD

(71) Applicant: SAMSUNG ELECTRONICS CO., LTD., Suwon-si (KR)

(72) Inventors: Myounghoon JUNG, Bucheon-si (KR); Kak NAMKOONG, Seoul (KR); Kunsun EOM, Seoul (KR); Yeolho LEE, Anyang-si (KR); Seongho CHO, Gwacheon-si (KR)

(73) Assignee: SAMSUNG ELECTRONICS CO., LTD., Suwon-si (KR)

(21) Appl. No.: 14/743,069

(22) Filed: Jun. 18, 2015

(30) Foreign Application Priority Data
Jan. 30, 2015 (KR) 10-2015-0015576

(51) Int. Cl. A61B 5/053; A61B 5/00

(52) U.S. Cl. CPC A61B 5/0537; A61B 5/443; A61B 5/7246; A61B 5/7278; A61B 5/4872; A61B 5/4875; A61B 5/681; A61B 2562/0214

(57) ABSTRACT

Body composition measuring apparatuses and methods are provided. An impedance of an electric circuit including a body is different according to body composition. A normal-state impedance of the electric circuit and an impedance less than the normal-state impedance are determined. The determined impedances are analyzed to acquire skin hydration. A body fat ratio is acquired from the normal-state impedance.

### CROSS-REFERENCE TO RELATED APPLICATION

This application claims priority from Korean Patent Application No. 10-2015-0015576, filed on Jan. 30, 2015 in the Korean Intellectual Property Office, the disclosure of which is incorporated herein in its entirety by reference.

### BACKGROUND

1. Field

Apparatuses and methods consistent with exemplary embodiments relate to body composition measuring apparatuses and methods.

2. Description of the Related Art

With the development of medicines and the increase in the average lifespan, there is a growing interest in health care. In this regard, there is also a growing interest in medical devices, and the variety of applications of medical devices has increased. Examples of such medical devices include medical devices used in hospitals or medical examination centers, small and medium sized medical devices installed in public institutions, and small medical devices and health care devices that are owned or carried by individuals.

A body composition measuring device is a type of health care device that measures body composition by using bioelectrical impedance analysis (BIA). BIA allows current to flow through the body by taking into account that the body has a combination of impedances, measures a voltage generated by body impedance, and measures the body impedance based on the current and the voltage.

In this case, the measurement of the body composition is performed while directly contacting an electrode with a part of the body. Thus, a contact resistance caused by the contact between the electrode and the body may influence the measurement of the body impedance.

### SUMMARY

One or more exemplary embodiments provide body composition measuring apparatuses and methods capable of measuring skin hydration by using a contact resistance between an electrode and a body.

Further, one or more exemplary embodiments provide body composition measuring apparatuses and methods capable of measuring skin hydration and body composition.

According to an aspect of an exemplary embodiment, there is provided a body composition measuring apparatus including: an electrode configured to be in contact with a body of a user; an impedance measuring unit connected to the electrode and configured to measure an impedance of the body; and a first determining unit configured to acquire skin hydration, based on a normal-state impedance of the body and a comparative impedance corresponding to a percentage of the normal-state impedance.

The first determining unit may include: a waveform detector configured to determine the normal-state impedance and the comparative impedance; and a skin hydration acquiring unit configured to determine the skin hydration based on the normal-state impedance and the comparative impedance.

The waveform detector may be further configured to determine a difference between a measurement time corresponding to the normal-state impedance and a measurement time corresponding to the comparative impedance, and the skin hydration acquiring unit may determine the skin hydration based on the determined difference.

The percentage may be about 95% to about 98%.

The body composition measuring apparatus may further include a second determining unit configured to acquire a body fat ratio based on the measured impedance of the body.

The electrode may include a first electrode configured to measure the skin hydration, and a second electrode configured to measure the body fat ratio, and the body composition measuring apparatus may further include an electrode switch configured to selectively connect the first and second electrodes to the impedance measuring unit.

The impedance measuring unit may measure a first impedance in a 4-point measuring mode and measure a second impedance in a 2-point measuring mode, and the second determining unit may include: a body impedance acquiring unit configured to determine the body impedance of the user based on the first impedance and the second impedance; and a body fat ratio acquiring unit configured to acquire the body fat ratio of the user based on the body impedance and body information of the user.

The electrode may further include a measuring mode switch configured to selectively connect the electrode to the impedance measuring unit in the 4-point measuring mode and the 2-point measuring mode.

The electrode may include a 4-point measuring electrode for 4-point measurement and a 2-point measuring electrode for 2-point measurement, and the impedance measuring unit may include: a first impedance measuring unit connected to the 4-point measuring electrode to measure the first impedance; and a second impedance measuring unit connected to the 2-point measuring electrode to measure the second impedance.

The electrode may further include a first electrode configured to measure the skin hydration, and the body composition measuring apparatus may further include an electrode switch configured to selectively connect the first electrode to one of the first and second impedance measuring units.

According to an aspect of another exemplary embodiment, there is provided a body composition measuring method including: measuring an impedance of a body of a user; determining a normal-state impedance of the body; determining a comparative impedance corresponding to a percentage of the normal-state impedance; and determining skin hydration based on the normal-state impedance and the comparative impedance.

The determining the skin hydration may include: determining a difference of a measurement time corresponding to the normal-state impedance and a measurement time corresponding to the comparative impedance; and determining the skin hydration based on the determined difference.

The percentage may be about 95% to about 98%.

The body composition measuring method may further include acquiring a body fat ratio based on the measured impedance.

The body fat ratio may be acquired based on the normal-state impedance.

The measuring the impedance may include selectively connecting an impedance measuring unit to a first electrode configured to measure the skin hydration and a second electrode configured to measure a body fat ratio.

The measuring the impedance may include: measuring a first impedance in a 4-point measuring mode; and measuring a second impedance in a 2-point measuring mode. The acquiring the body fat ratio may include: determining the body impedance based on the first impedance and the second impedance; and acquiring the body fat ratio of the user based on the body impedance and body information of the user.

The measuring the impedance may include selectively switching a connection mode of an electrode and an impedance measuring unit to the 4-point measuring mode and the 2-point measuring mode.

### BRIEF DESCRIPTION OF THE DRAWINGS

The above and/or other aspects will be more apparent by describing certain exemplary embodiments, with reference to the accompanying drawings, in which:

FIG. 1 is a configuration diagram of a body composition measuring apparatus according to an exemplary embodiment;

FIG. 2 is a circuit diagram of the body composition measuring apparatus of FIG. 1;

FIG. 3 is a circuit diagram of an impedance measuring unit employing a 4-point measuring mode using four electrodes, according to an exemplary embodiment;

FIG. 4 is a graph of impedance with respect to degrees of dryness;

FIG. 5 is a graph of a relationship between skin hydration and a time difference between 1,500 unit time and a time when an impedance corresponding to 98% of an impedance at 1,500 unit time is measured in FIG. 4;

FIGS. 6 and 7 are perspective views of a body composition measuring apparatus, according to an exemplary embodiment;

FIG. 8A is a flowchart of a skin hydration measuring method according to an exemplary embodiment;

FIG. 8B is a flowchart of a skin hydration measuring method according to another exemplary embodiment;

FIG. 9 is a configuration diagram of a body composition measuring apparatus capable of measuring skin hydration and a body fat ratio, according to an exemplary embodiment;

FIG. 10 is a circuit diagram of the body composition measuring apparatus of FIG. 9;

FIG. 11 is a circuit diagram of the body composition measuring apparatus of FIG. 9 in which the impedance measuring unit is switched to a 4-point measuring mode by a measuring mode switch;

FIG. 12 is a circuit diagram of the body composition measuring apparatus of FIG. 9 in which the impedance measuring unit is switched to a 2-point measuring mode by the measuring mode switch;

FIG. 13 is a configuration diagram of a body composition measuring apparatus according to another exemplary embodiment;

FIG. 14 is a circuit diagram illustrating an example where an electrode switch is applied to the body composition measuring apparatus of FIG. 10;

FIG. 15 is a circuit diagram illustrating an example where an electrode switch is applied to the body composition measuring apparatus of FIG. 10;

FIG. 16 is a circuit diagram illustrating an example where an electrode switch is applied to the body composition measuring apparatus of FIG. 11;

FIG. 17 is a perspective view of an implementation of the body composition measuring apparatus illustrated in FIG. 10;

FIG. 18 is a perspective view of an implementation of the body composition measuring apparatus illustrated in FIG. 11; and

FIGS. 19A and 19B are flowcharts of body composition measuring methods according to exemplary embodiments.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

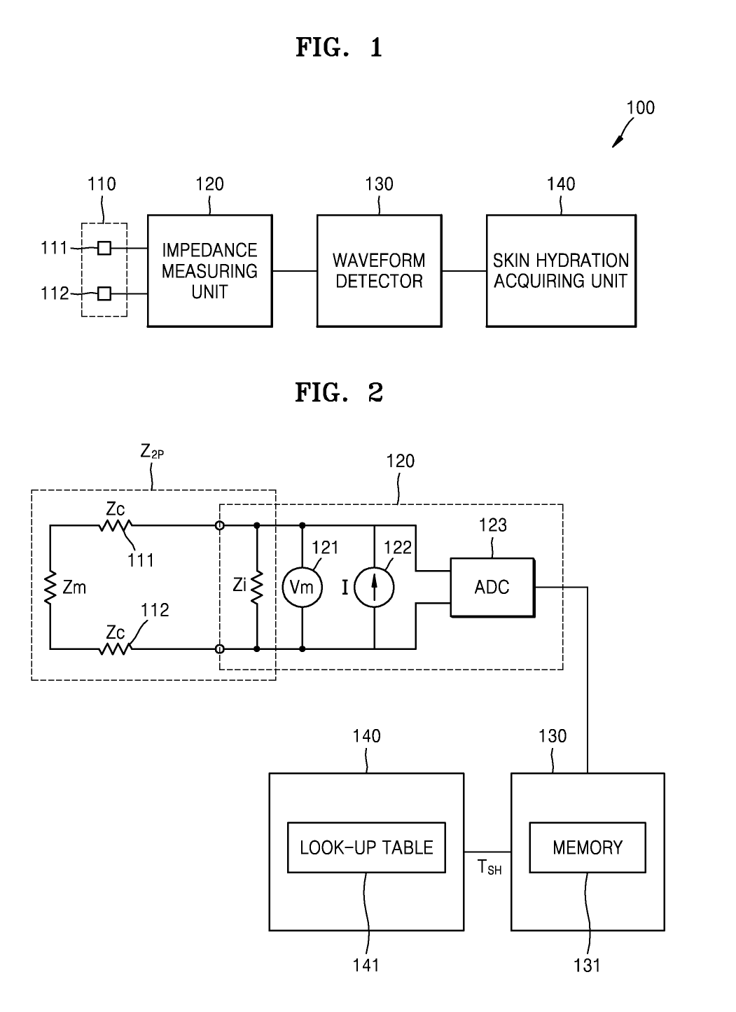

FIG. 1 is a configuration diagram of a body composition measuring apparatus 100 according to an exemplary embodiment. FIG. 2 is a circuit diagram of the body composition measuring apparatus 100 of FIG. 1. Referring to FIGS. 1 and 2, the body composition measuring apparatus 100 may include an electrode 110, an impedance measuring unit 120, a waveform detector 130, and a skin hydration acquiring unit 140.

The electrode 110 may include two or more electrodes. For example, as illustrated in FIG. 1, the electrode 110 includes two electrodes 111 and 112. When the body composition measuring apparatus 100 is a wearable device such as a smart watch, the electrodes 111 and 112 may come in contact with a user's body when the user wears the wearable device. When the user wears the wearable device, one of the electrodes 111 and 112 may come in contact with the user's body and the other may come in contact with the user's body due to the action of the user. Alternatively, both electrodes 111 and 112 may come in contact with the user's body due to the action of the user.

The impedance measuring unit 120 may measure an impedance of an electric circuit, including the body, by using bioelectrical impedance analysis (BIA). The impedance measuring unit 120 allows current to flow through the electrodes 111 and 112, measures a voltage generated by the current, and measures the impedance based on the current and the voltage.

FIG. 2 is a circuit diagram of the impedance measuring unit 120 that employs a 2-point measuring mode using the two electrodes 11 and 112. Zc is a contact impedance between the electrodes 111 and 112 and the user's body. Zm is a body impedance of the user's body. Zi is an impedance of an analog front end (AFE) which is an analog circuit. A current source 122 may apply a constant current to the body through the electrodes 111 and 112. A voltage, which is measured by a voltmeter 121, may be output to an analog-to-digital converter (ADC) 123. The ADC 123 may convert a voltage input in the form of an analog signal into a digital signal. Since the magnitude of the current is fixed, the voltage Vi, which is measured by the impedance measuring unit 120, is proportional to an impedance Z_{2P}. The impedance Z_{2P} may be calculated by dividing the voltage Vi by a current. The impedance Z_{2P} may be expressed as Equation 1 below.

$${Z}_{2 P} = f ( Zm , Zc , Zi ) = \frac{1}{\frac{1}{Zm + 2 Zc} + \frac{1}{Zi}} \qquad [ Equation 1 ]$$

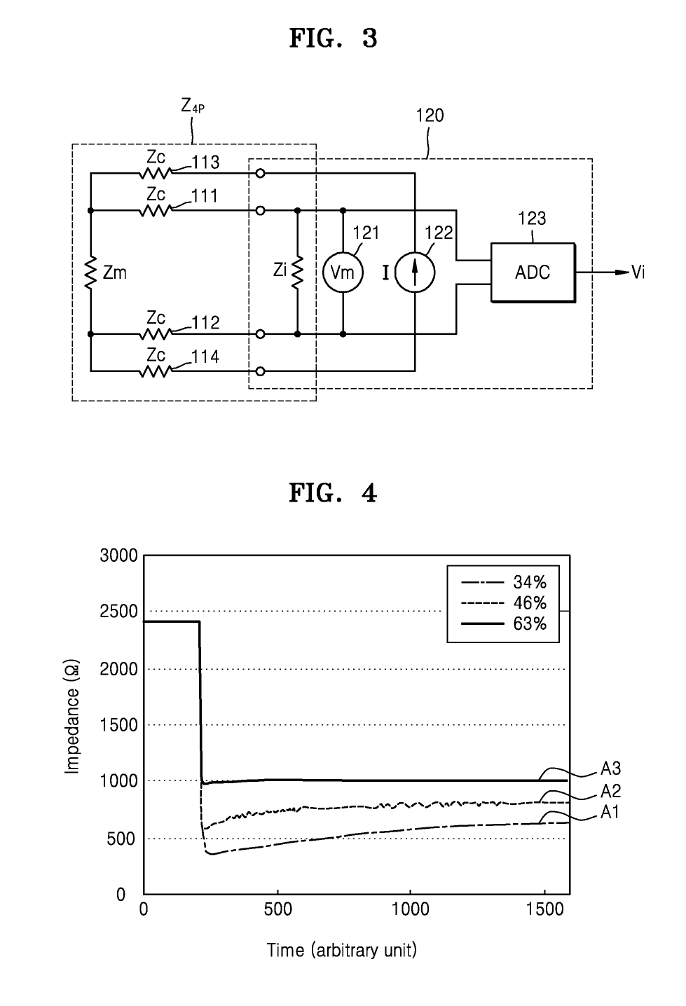

The number of the electrodes provided in the electrode 110 is not limited to two. For example, four electrodes may be provided. FIG. 3 is a circuit diagram of the impedance measuring unit 120 employing a 4-point measuring mode using four electrodes 111 to 114, according to an exemplary embodiment. Referring to FIG. 3, a current source 122 may be connected to the two electrodes 113 and 114, and a voltmeter 121 may measure a voltage between the other electrodes 111 and 112. The voltage, which is measured by the voltmeter 121, may be output to an ADC 123. The ADC 123 may convert a voltage input in the form of an analog signal into a digital signal. Since a magnitude of a current is fixed, a voltage Vi, which is measured by an impedance measuring unit 120, is proportional to an impedance Z_{4P}. The impedance Z_{4P} may be calculated by dividing the voltage Vi by a current. The impedance Z_{4P} may be expressed as Equation 2 below.

$${Z}_{4 P} = f ( Zm , Zc , Zi ) = Zm \frac{1}{1 + \frac{Zm + 2 Zc}{Zi}} \qquad [ Equation 2 ]$$

As shown in Equations 1 and 2, Z_{2P} and Z_{4P} are determined by Zm, Zc, and Zi. Zi is determined according to characteristics of the AFE. Thus, Z_{2P} and Z_{4P} are dependent on Zm and Zc. Since Zm is the body impedance and is determined according to the body composition of the user, Zm is a fixed value for the same user. For example, if a value measured when the body (e.g., a finger) is in contact with the electrodes 111 and 112 does not fluctuate and is in a stable normal state, a very small amount of current flows through the electrodes 111 and 112. Thus, a voltage applied between the finger and the electrodes 111 and 112 is substantially 0 V. However, if the finger is in contact with the electrodes 111 and 112 at the time of starting measurement, the voltage applied to the electrodes 111 and 112 changes instantaneously and a part of the voltage is applied between the body and the electrodes 111 and 112. This is referred to as a transient state. In the transient state, the voltage measured by the impedance measuring unit 120 changes with time. When the voltage becomes a normal state, the voltage is saturated and stabilized.

Assuming that V0 is a voltage between the finger and the electrodes 111 and 112 at the moment that the finger is in contact with the electrodes 111 and 112, R is a resistance component of a contact impedance Zc, C is a capacitance component, and t is the time that elapses from the moment that the finger is in contact with the electrodes 111 and 112, the voltage applied between the finger and the electrodes 111 and 112 at the time t may be expressed as Equation 3 below.

$$V = {V}_{0} {e}^{- \frac{t}{RC}} \qquad [ Equation 3 ]$$

As shown in Equation 3, the time t necessary for switching from the transient state to the normal state is related to the resistance component R and the capacitance component C of the contact impedance Zc. If the skin hydration changes, the resistance component R and the capacitance component C of the skin changes. Accordingly, it is possible to determine the skin hydration. When the skin is dry, the time it takes to switch to the normal state is increased, and when the skin is wet, the time it takes to switch to the normal state is shortened.

FIG. 4 is a graph of impedance with respect to degrees of dryness. A1, A2, and A3 are impedances measured when the skin (finger) hydration is 34%, 46%, and 65%, respectively. As shown in FIG. 3, as the skin hydration is lower, the time to saturate the impedance to the normal state is increased.

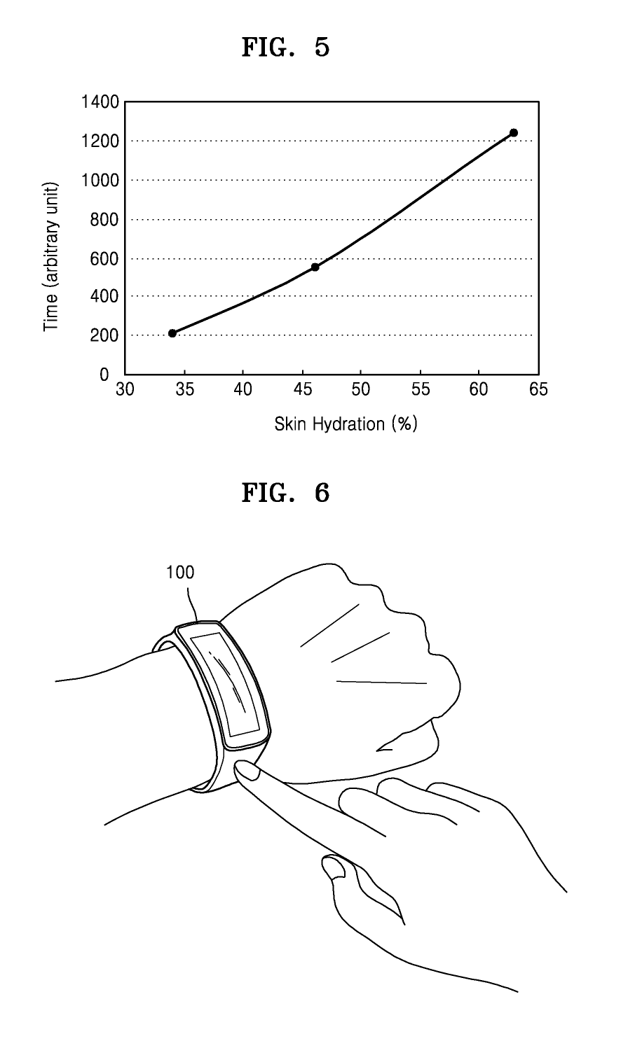

FIG. 5 is a graph of a relationship between skin hydration and a time difference between 1,500 unit time and a time when an impedance corresponding to 98% of an impedance at 1,500 unit time is measured in FIG. 4. Referring to FIG. 5, as the skin hydration lowers, a change rate of the impedance is increased. It takes a short time for the impedance to change by 2%. As the skin hydration gets higher, a slope of the impedance is reduced, and thus, it takes a long time for the impedance to change by 2%.

Therefore, the skin hydration may be obtained by storing a measurement time and a measurement voltage (or a measurement impedance corresponding to the measurement voltage) in a memory and calculating the time taken for the measurement voltage (or the measurement impedance) to change at a constant rate. For example, the skin hydration may be obtained by determining a measurement impedance of a normal state, that is, a normal-state impedance, and a comparative impedance corresponding to n % of the normal-state impedance and calculating the time that elapses to change from the comparative impedance to the normal-state impedance. The normal-state impedance may be an impedance value measured when a slope (ΔZ/ΔT) of the measurement impedance with respect to time becomes, for example, 2 Ω/sec or less. A value of n, which determines the comparative impedance, may be, for example, about 95% to about 98%.

When the measurement of the skin hydration is started, the impedance measuring unit 120 may supply a constant current to the electric circuit including the body and output to the waveform detector 130 a measurement voltage Vi corresponding to an impedance of the electric circuit.

The waveform detector 130 may store the measurement time and the measurement voltage Vi in the memory 131. Alternatively, the waveform detector 130 may store the measurement impedance in the memory 131, the measurement impedance being calculated by dividing the measurement voltage Vi by the supplied current. When the measurement voltage Vi (or the measurement impedance) is saturated, the waveform detector 130 may determine the measurement voltage Vi (or the measurement impedance) as the normal-state voltage (impedance). Then, the waveform detector 130 may determine a comparative voltage (impedance) corresponding to, for example, 98% of the normal-state voltage (impedance) and output to the skin hydration acquiring unit 140 the time T_{SH} that elapses until the voltage (impedance) changes from the comparative voltage (impedance) to the normal-state voltage (impedance). The skin hydration acquiring unit 140 may determine the skin hydration by using the elapsed time T_{SH}. For example, the skin hydration acquiring unit 140 may acquire the skin hydration with respect to the elapsed time T_{SH} by using a look-up table 141. The look-up table 141 may prestore values of the skin hydration that are calculated according to the elapsed time T_{SH}. The look-up table 141 may be configured to receive the elapsed time T_{SH} and output the skin hydration. The skin hydration acquiring unit 140 may calculate the skin hydration by using the elapsed time T_{SH}, based on a preset calculation equation.

In the body composition measuring apparatuses 100 according to the above-described exemplary embodiments, the skin hydration may be measured by measuring the impedance of the electric circuit.

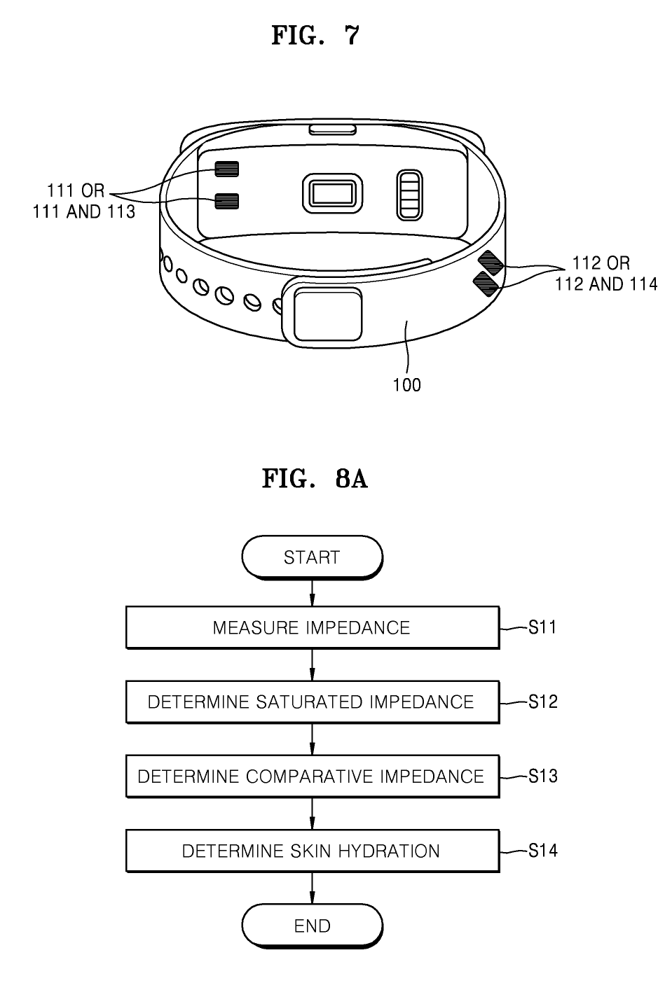

FIGS. 6 and 7 are perspective views of a body composition measuring apparatus 100, according to an exemplary embodiment. Referring to FIGS. 6 and 7, the body composition measuring apparatus 100 may be a wearable device, such as a smart watch wearable on his or her wrist or a smart ring wearable on a finger. In a 2-point measuring mode, electrodes 111 and 112 may be respectively disposed inside and outside the body composition measuring apparatus 100. Thus, when a user wears the body composition measuring apparatus 100 on the wrist, the electrode 111 may be in contact with a skin surface of a wrist and the user may be able to touch the electrode 112 by his or her finger of the other wrist, as illustrated in FIG. 6. In a 4-point measuring mode, electrodes 111 and 113 and electrodes 112 and 114 may be respectively disposed inside and outside the body composition measuring apparatus 100. Thus, when a user wears the body composition measuring apparatus 100 on his or her wrist, the electrodes 111 and 113 may be in contact with a skin of the wrist and the user may be able to touch the electrodes 112 and 114 by his or her finger of the other wrist, as illustrated in FIG. 6.

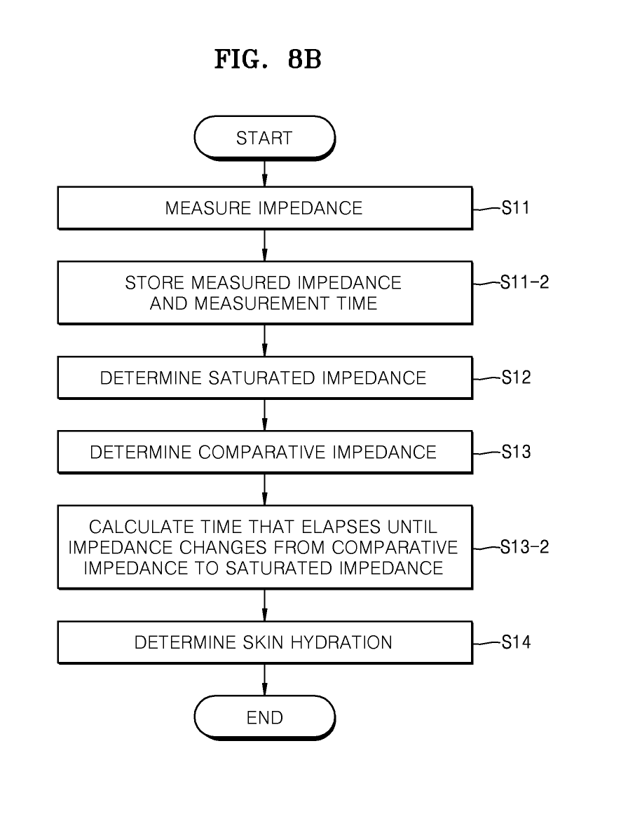

FIGS. 8A and 8B are flowcharts of skin hydration measuring methods according to exemplary embodiments. The skin hydration measuring methods according to exemplary embodiments will be described below with reference to FIGS. 1, 8A, and 8B.

In operation S11, the impedance of the electric circuit including the body may be measured. The measurement of the impedance for measuring the skin hydration may be started by wearing the body composition measuring apparatus 100 on a wrist and pressing a button or the like. For example, the body composition measuring apparatus 100 may be worn on a wrist of a left hand. In this case, the electrode 111 or the electrodes 111 and 113 may be in contact with the body, and a measurement start command may be input to the body composition measuring apparatus 100 by pressing the button or the like by using the finger of the right hand. The finger of the right hand may come into contact with the electrode 112 or the electrodes 112 and 114. The impedance measuring unit 120 may supply a constant current to the electric circuit including the body and output to the waveform detector 130 a measurement voltage Vi corresponding to the impedance of the electric circuit.

In operation S12, the waveform detector 130 may determine the normal-state impedance. For example, when the slope of the measurement voltage Vi (or the measurement impedance) with respect to the measurement time is a predetermined value or less, the measurement voltage (Vi) (or the measurement impedance) at that time may be determined as the normal-state voltage (impedance). In addition, the measurement voltage Vi (or the measurement impedance) after a predetermined time has elapsed from the start of the measurement may be determined as the normal-state voltage (impedance).

In operation S13, the comparative voltage (impedance) corresponding to n % of the normal-state voltage (impedance) may be determined.

In operation S14, the skin hydration may be determined based on the normal-state voltage (impedance) and the comparative voltage (impedance).

For example, as illustrated in FIG. 8B, in operation S11-2, the measurement voltage Vi (or the measurement impedance obtained by dividing the measurement voltage Vi by the supplied current) and the measurement time may be stored in the memory 131. In addition, in operation S13-2, the time T_{SH} that elapses until the voltage (impedance) changes from the comparative voltage (impedance) to the normal-state voltage (impedance) may be calculated. The elapsed time T_{SH} may be a difference between the measurement time of the normal-state voltage (impedance) and the measurement time of the comparative voltage (impedance). In operation S14, the skin hydration may be determined from the elapsed time T_{SH} by the preset calculation equation or may be determined by using the look-up table 141.

The determined skin hydration may be provided as visual information to a user through a display.

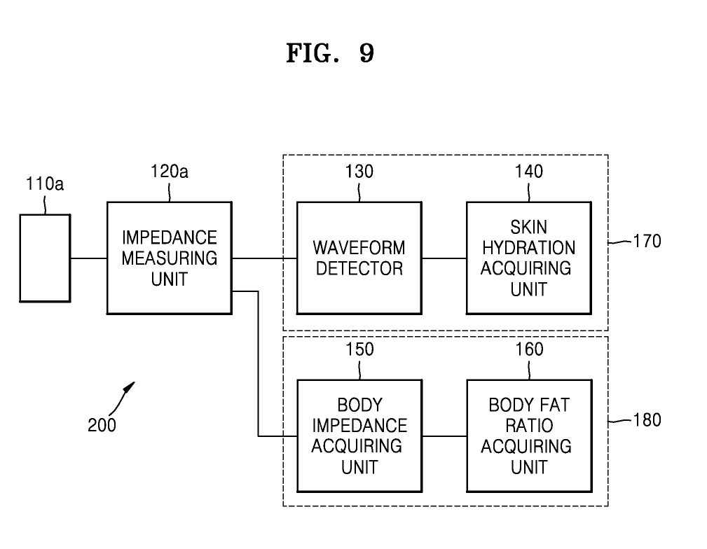

FIG. 9 is a configuration diagram of a body composition measuring apparatus 200 capable of measuring skin hydration and a body fat ratio, according to an exemplary embodiment. Referring to FIG. 9, the body composition measuring apparatus 200 may include an electrode 110a, an impedance measuring unit 120a, a first determining unit 170 configured to acquire the skin hydration, and a second determining unit 180 configured to acquire the body fat ratio. The first determining unit 170 may include a waveform detector 130 and a skin hydration acquiring unit 140. The second determining unit 180 may include a body impedance acquiring unit 150 and a body fat ratio acquiring unit 160. According to the present exemplary embodiment, the electrode 110a may be used to measure the skin hydration and the body fat ratio. The configurations and operations of the waveform detector 130 and the skin hydration acquiring unit 140 may be substantially identical to those described with reference to FIGS. 1 to 3. The waveform detector 130 and the skin hydration acquiring unit 140 may be implemented with a computer processor, and the body impedance acquiring unit 150 and the body fat ratio acquiring unit 160 may be implemented with another computer processor. However, the present exemplary embodiment is not limited thereto, and the body impedance acquiring unit 150 and the body fat ratio acquiring unit 160 may be implemented with the same computer processor as the waveform detector 130 and the skin hydration acquiring unit 140.

The impedance measuring unit 120a may be substantially identical to the impedance measuring unit 120 described with reference to FIG. 2 or 3.

The waveform detector 130 may determine the normal-state impedance and the comparative impedance from the measurement impedance during the transient state, and the skin hydration acquiring unit 140 may determine the skin hydration based on the time that elapses until the impedance changes from the comparative impedance to the normal-state impedance.

The body impedance acquiring unit 150 may acquire the body impedance Zm from the impedance value, Z_{4P} or Z_{2P} after the saturation of the measurement impedance. For example, as described above, although the body impedance Zm depends on Zc, Zc may be assumed as “0” if Zc is much smaller than Zm, as shown in Equations 1 and 2 above. Therefore, Zm may be calculated. In addition, the body impedance acquiring unit 150 may acquire Zm from the look-up table that prestores Zm corresponding to Z_{4P} or Z_{2P}.

Z_{4P} or Z_{2P} may be an average of values obtained from measuring twice or more and may be an average of measured values sampled at different points of time during the normal state.

In addition, before the body impedance Zm is acquired, a process of determining the validity of Z_{4P} or Z_{2P} may be performed. During the measurement of the impedance, an impedance measured without contact between the body and the electrode is invalid. Therefore, the body composition measuring apparatus 200 may determine the validity of the measured impedance by checking the state occurring during the measurement of the impedance. As another example, the validity of the impedance may be determined according to the magnitude of the measured impedance. That is, the validity of the impedance may be determined according to whether the measured impedance is within an impedance range that is measurable from the body.

The body impedance Zm is an impedance necessary for calculating the body fat ratio and is changed according to body characteristics. The body fat ratio acquiring unit 160 may acquire the body fat ratio of the user by using the body impedance Zm and the body information of the user. The body information of the user may be received from the user. The body information of the user may be age, height, or weight of the user.

According to such a configuration, the skin hydration may be acquired from the impedance measured during the transient state and the body fat ratio may be determined based on the impedance measured during the normal state. Since the skin hydration and the body fat ratio are determined based on the impedance measured by the common impedance measuring unit 120a, it is possible to implement the small-sized body composition measuring apparatus 200 capable of measuring the skin hydration and the body fat ratio. In addition, since it is possible to reduce the size of the body composition measuring apparatus 200, the body composition measuring apparatus 200 may be implemented in the form of a wearable device or may be integrated into a wearable device such as a smart watch.

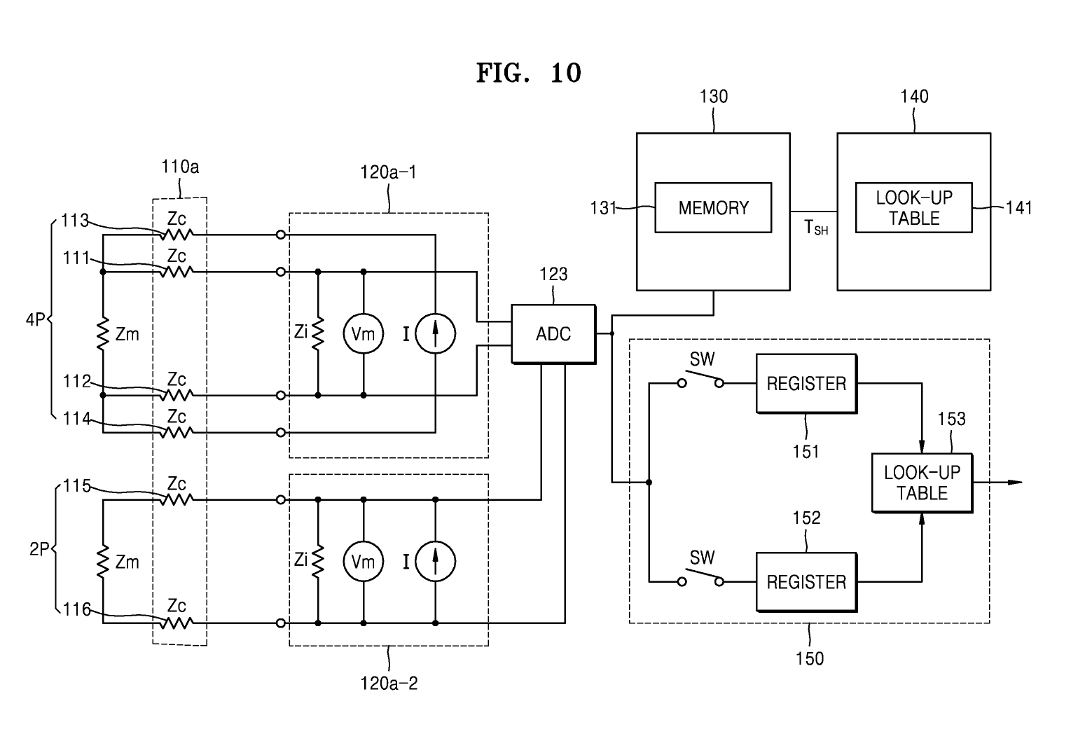

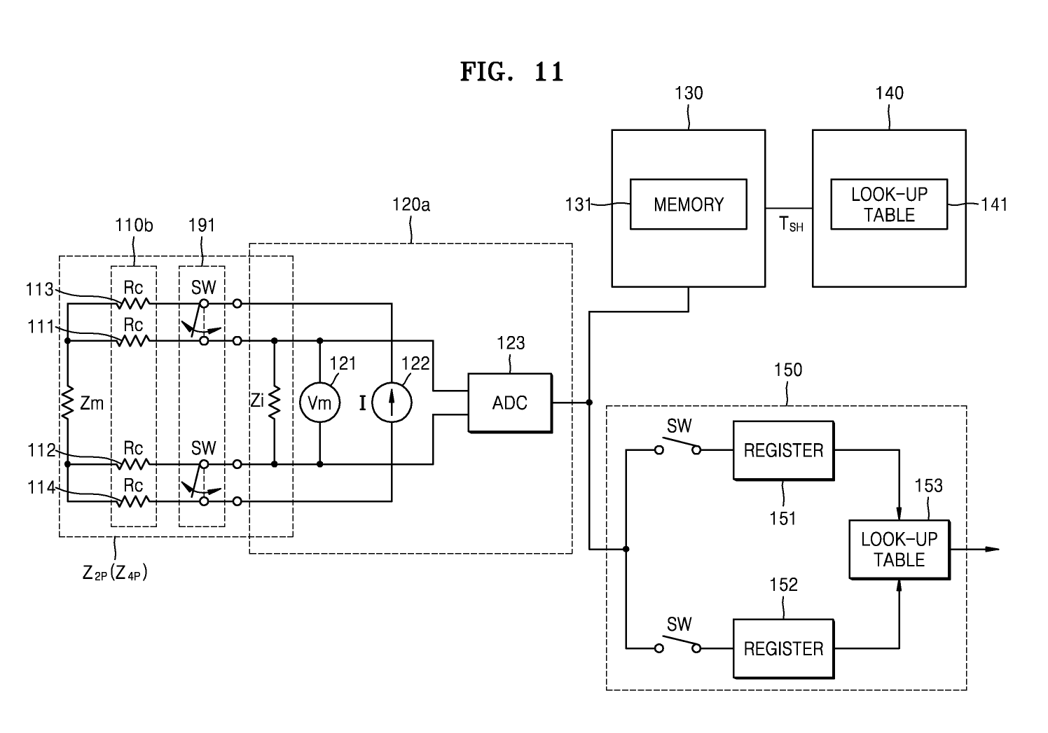

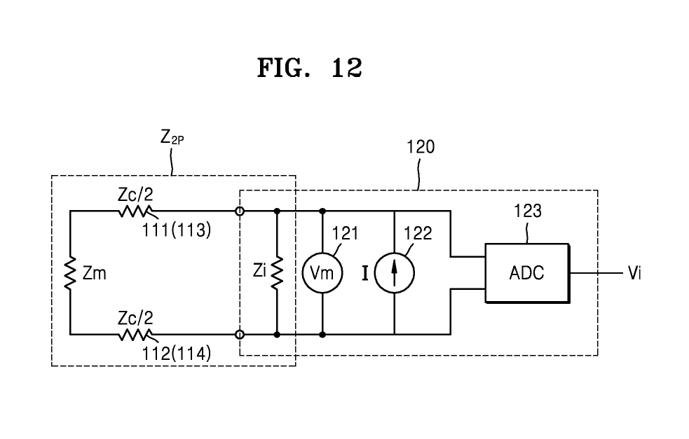

FIG. 10 is a circuit diagram of the impedance measuring unit 120a of FIG. 9. In the present exemplary embodiment, the electrode 110a may include a 4-point measuring electrode 4P and a 2-point measuring electrode 2P. The 4-point measuring electrode 4P may include current electrodes 113 and 114 and voltage electrodes 111 and 112.

The impedance measuring unit 120a may measure first and second impedances Z_{4P} and Z_{2P} by measuring the voltage corresponding to the impedance of the electric circuit including the body in a 2-point measuring mode and a 4-point measuring mode. The impedance measuring unit 120a may include a first impedance measuring unit 120a-1 configured to measure the first impedance Z_{4P} in the 4-point measuring mode by using the four electrodes 111 to 114, and a second impedance measuring unit 120a-2 configured to measure the second impedance Z_{2P} in the 2-point measuring mode by using the two electrodes 115 and 116.

The first impedance measuring unit 120a-1 may measure the first impedance Z_{4P} of the electric circuit including the body by using the 4-point measuring mode. The first impedance measuring unit 120a-1 may be substantially identical to the impedance measuring unit 120 of FIG. 3. The first impedance measuring unit 120a-1 may apply a constant current through the electrodes 113 and 114 and measure the first impedance Z_{4P} through the electrodes 111 and 112. The first impedance measuring unit 120a-1 may measure the voltage Vi applied to the electrodes 111 and 112. The voltage Vi is proportional to the first impedance Z_{4P}. The first impedance Z_{4P} may be obtained by dividing the voltage Vi by the supplied current. The first impedance Z_{4P} may be expressed as Equation 2 (first relational expression) above.

The second impedance measuring unit 120a-2 may measure the second impedance Z_{2P} of the electric circuit including the body in the 2-point measuring mode. The second impedance measuring unit 120a-2 may be substantially identical to the impedance measuring unit 120 of FIG. 2. The second impedance measuring unit 120a-2 may measure the second impedance Z_{2P} in the 2-point measuring mode by using the electrodes 115 and 116. The 2-point measuring mode may be substantially identical to that described with reference to FIG. 2. The second impedance measuring unit 120a-2 may apply a constant current through the electrodes 115 and 116 and measure the second impedance Z_{2P} through the electrodes 115 and 116. The second impedance measuring unit 120a-2 may measure the voltage Vi applied to the electrodes 115 and 116. The voltage Vi is proportional to the second impedance Z_{2P} The second impedance Z_{2P} may be obtained by dividing the voltage Vi by the supplied current. The second impedance Z_{2P} may be expressed as Equation 1 (second relational expression) above.

The operations of the waveform detector 130 and the skin hydration acquiring unit 140 are substantially identical to those described above. That is, the waveform detector 130 may store the measurement voltage (or the measurement impedance) output from the first impedance measuring unit 120a-1 or the second impedance measuring unit 120a-2 in the memory 131, determine the normal-state voltage (impedance) and the comparative voltage (impedance) corresponding to n % of the normal-state voltage (impedance), and output to the skin hydration acquiring unit 140 the time T_{SH} that elapses until the voltage (impedance) changes from the comparative voltage (impedance) to the normal-state voltage (impedance). The skin hydration acquiring unit 140 may determine the skin hydration by using the elapsed time T_{SH}.

When the first and second impedances Z_{4P} and Z_{2P} are measured, the body impedance acquiring unit 150 may acquire the body impedance Zm determined according to the measured first and second impedances Z_{4P} and Z_{2P}.

The first and second impedances Z_{4P} and Z_{2P} are impedances in the normal state in which the measurement is stabilized. The first and second impedances Z_{4P} and Z_{2P} may be an average of values obtained from measuring twice or more. The first and second impedances Z_{4P} and Z_{2P} may be an average of measured values sampled at different points of time during the normal state.

The body impedance acquiring unit 150 may calculate the body impedance Zm through the first and second relational expressions, regardless of the contact impedance Zc. That is, referring to Equations 1 and 2, Z_{4P} and Z_{2P} are obtained by dividing the measured voltage Vi by the supplied current, and Zi is determined according to characteristics of AFE. Therefore, Zm and Zc may be calculated by combining Equations 1 and 2. That is, even when Zc is unknown or cannot be calculated, Zm may be calculated by eliminating Zc.

The body impedance acquiring unit 150 may acquire the body impedance Zm by using a look-up table 153. The body impedance acquiring unit 150 may include two switches SW, registers 151 and 152, and the look-up table 153. The two switches SW may be turned on and off according to the measuring mode. For example, in the 4-point measuring mode, the upper switch SW may be closed and the lower switch SW may be opened. For example, in the 2-point measuring mode, the upper switch SW may be closed and the lower switch SW may be opened. In the 4-point measuring mode, the measurement voltage (impedance) may be stored in the upper register 151. In the 2-point measuring mode, the measurement voltage (impedance) may be stored in the lower register 152.

The look-up table 153 may receive the values of the measurement voltages (impedances) stored in the registers 151 and 152 and output the body impedance Zm. When the look-up table 153 receives the values of two measurement voltages (impedances), the look-up table 153 may determine a corresponding value. The determined value is the body impedance Zm. For example, the look-up table 153 may store a 2×2 table with respect to the values of the two voltages (impedances). A horizontal axis may be the first impedance Z_{4P} or the corresponding measurement voltage, and a vertical axis may be the second impedance Z_{2P} or the corresponding measurement voltage. Therefore, when the value of the horizontal axis and the value of the vertical axis are determined, the look-up table 153 may determine the body impedance Zm corresponding to the values of the horizontal axis and the value of the vertical axis. The look-up table 153 may output the determined value to the body fat ratio acquiring unit 160.

The body impedance Zm is an impedance necessary for calculating the body fat ratio and is changed according to body characteristics. The body fat ratio acquiring unit 160 may acquire the body fat ratio of the user by using the body impedance Zm and the body information of the user. The body information of the user may be received from the user. The body information of the user may be age, height, or weight of the user.

FIG. 11 is a circuit diagram of the impedance measuring unit 120a of FIG. 9. Referring to FIG. 11, the electrode 110b may include four electrodes 111 to 114. The impedance measuring unit 120a may measure the first impedance Z_{4P} of the user in the 4-point measuring mode and may measure the second impedance Z_{2P} of the user in the 2-point measuring mode. To this end, a measuring mode switch 191 may be provided. The measuring mode switch 191 may connect the four electrodes 111 to 114 to the impedance measuring unit 120a by switching to the 4-point measuring mode and the 2-point measuring mode.

FIG. 11 is a circuit diagram in which the impedance measuring unit 120a is switched to the 4-point measuring mode by the measuring mode switch 191. The impedance measuring unit 120a may apply a constant current through the electrodes 113 and 114 and measure the first impedance Z_{4P} through the electrodes 111 and 112. The impedance measuring unit 120a may measure the voltage Vi applied to the electrodes 111 and 112. The voltage Vi is proportional to the first impedance Z_{4P}. The first impedance Z_{4P} may be obtained by dividing the voltage Vi by the supplied current. The first impedance Z_{4P} may be expressed as Equation 2 (first relational expression) above.

In order to measure the second impedance Z_{2P}, the measuring mode switch 191 may connect the current electrodes 113 and 114 to the voltage electrodes 111 and 112, respectively. That is, the measuring mode switch 191 may connect the electrode 113 to the electrode 111 and connect the electrode 112 to the electrode 114. The current electrodes 113 and 114 are electrodes to which the constant current is applied in the 4-point measuring mode, and the voltage electrodes 111 and 112 are electrodes connected to the voltmeter 121 in the 4-point measuring mode. The connected electrodes 113 and 111 become one electrode and the connected electrodes 114 and 112 become another electrode. The impedance measuring unit 120a may measure the second impedance Z_{2P} in the 2-point measuring mode by using the connected electrodes 113 and 111 and the connected electrodes 114 and 112. FIG. 12 is a circuit diagram illustrating a state where the impedance measuring unit 120a is switched to the 2-point measuring mode by the measuring mode switch 191. The second impedance Z_{2P} according to the circuit diagram of FIG. 12 may be expressed as Equation 4 (second relational expression) above.

$${Z}_{f 2} = f ( Zm , Zc , Zi ) = \frac{1}{\frac{1}{Zm + Zc} + \frac{1}{Zi}} \qquad [ Equation 4 ]$$

The operations of the waveform detector 130 and the skin hydration acquiring unit 140 are substantially identical to those described above.

The operations of the body impedance acquiring unit 150 and the body fat acquiring unit 160 are substantially identical to those described above. The body impedance acquiring unit 150 may calculate the body impedance Zm through the first and second relational expressions, regardless of the contact impedance Zc, or may acquire the body impedance Zm by using the look-up table 153.

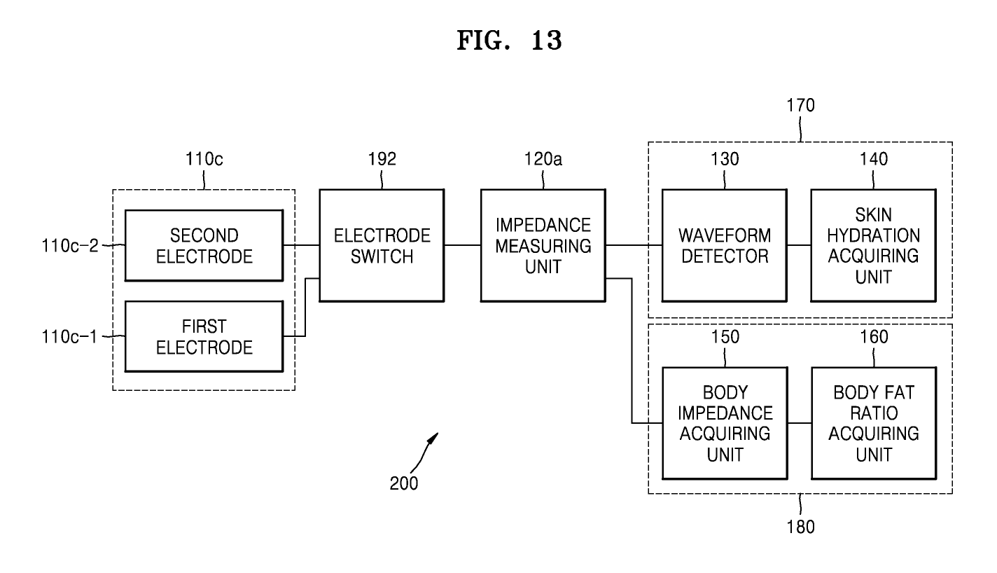

FIG. 13 is a configuration diagram of a body composition measuring apparatus 200 according to another exemplary embodiment. Referring to FIG. 13, an electrode 110c may include a first electrode 110c-1 configured to measure skin hydration, and a second electrode 110c-2 configured to measure a body fat ratio. Since the skin hydration and the body fat ratio are measured by using an impedance, an impedance measuring unit 120a may be commonly used. To this end, an electrode switch 192 may be provided. The electrode switch 192 may be disposed between the electrode 110c and the impedance measuring unit 120a and selectively connect the first and second electrodes 110c-1 and 110c-2 to the impedance measuring unit 120a. Therefore, the first and second electrodes 100c-1 and 100c-2 suitable for the measurement of the skin hydration and the body fat ratio may be applied, thus improving the reliability of measurement.

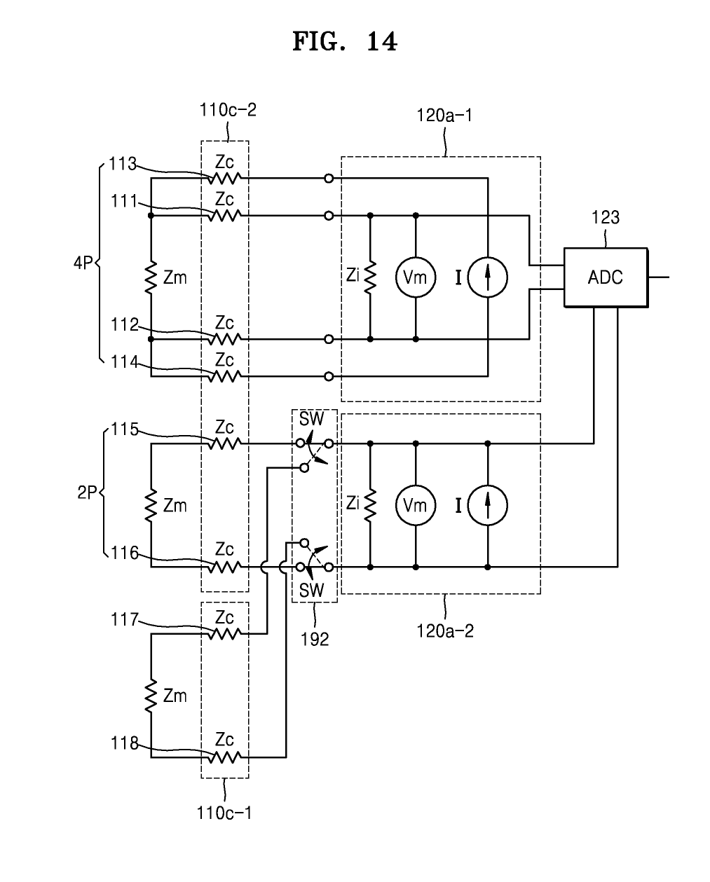

FIG. 14 is a circuit diagram illustrating an example where the electrode switch 192 is applied to the body composition measuring apparatus 200 of FIG. 10. Referring to FIG. 14, the first electrode 110c-1 may include electrodes 117 and 118. The first electrode 110a-1 may be selectively connected to the second impedance measuring unit 120a-2 by the electrode switch 192. For example, the two switches SW may be switched to positions (indicated by a solid line) for connecting the electrodes 115 and 116 to the second impedance measuring unit 120a-2, and may be switched to positions (indicated by a dashed line) for connecting the electrodes 117 and 118 to the second impedance measuring unit 120a-2.

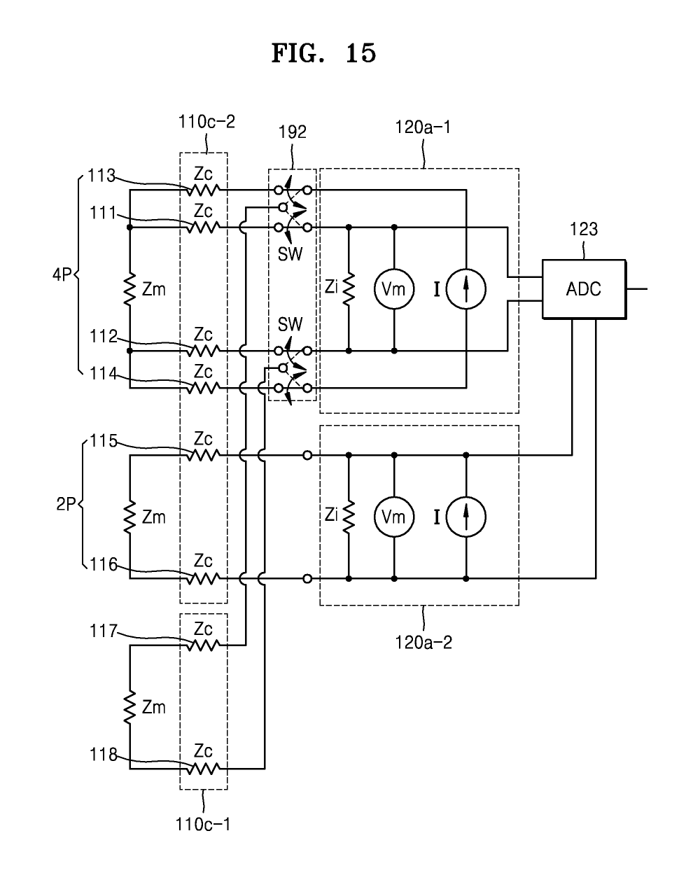

FIG. 15 is a circuit diagram illustrating an example where the electrode switch 192 is applied to the body composition measuring apparatus 200 of FIG. 10. Referring to FIG. 15, the first electrode 110c-1 may be selectively connected to the first impedance measuring unit 120a-1 by the electrode switch 192. For example, the two switches SW may be switched to positions (indicated by a solid line) for connecting the electrodes 111 and 113 and the electrodes 112 and 114 to the first impedance measuring unit 120a-1, and may be switched to positions (indicated by a dashed line) for connecting the electrodes 117 and 118 to the first impedance measuring unit 120a-1.

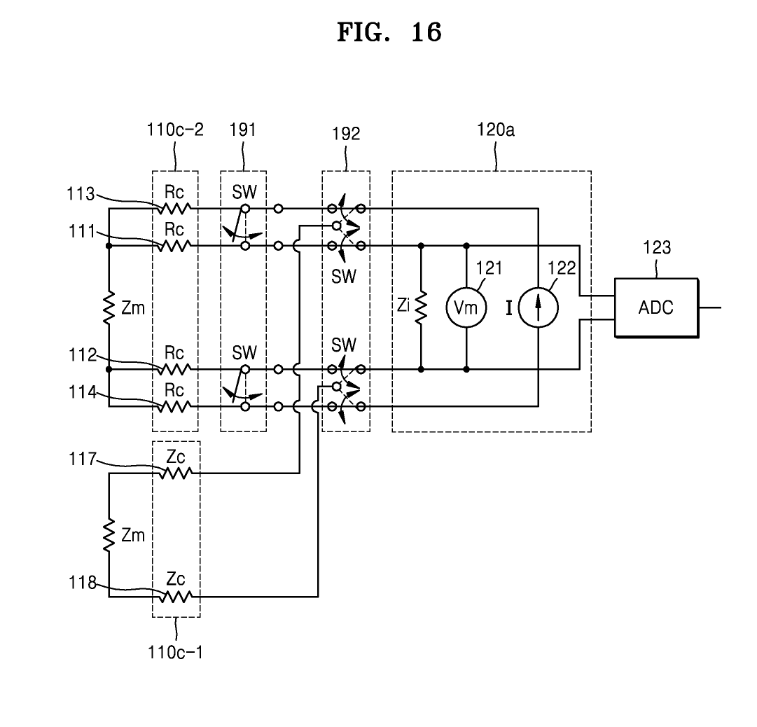

FIG. 16 is a circuit diagram illustrating an example where the electrode switch 192 is applied to the body composition measuring apparatus 200 of FIG. 11. Referring to FIG. 16, the first electrode 110c-1 may be selectively connected to the impedance measuring unit 120a by the electrode switch 192. For example, the two switches SW may be switched to positions (indicated by a solid line) for connecting the electrodes 111 and 113 and the electrodes 112 and 114 to the impedance measuring unit 120a, and may be switched to positions (indicated by a dashed line) for connecting the electrodes 117 and 118 to the impedance measuring unit 120a. The second electrode 110c-2 may be substantially identical to the electrode 110b of FIG. 11.

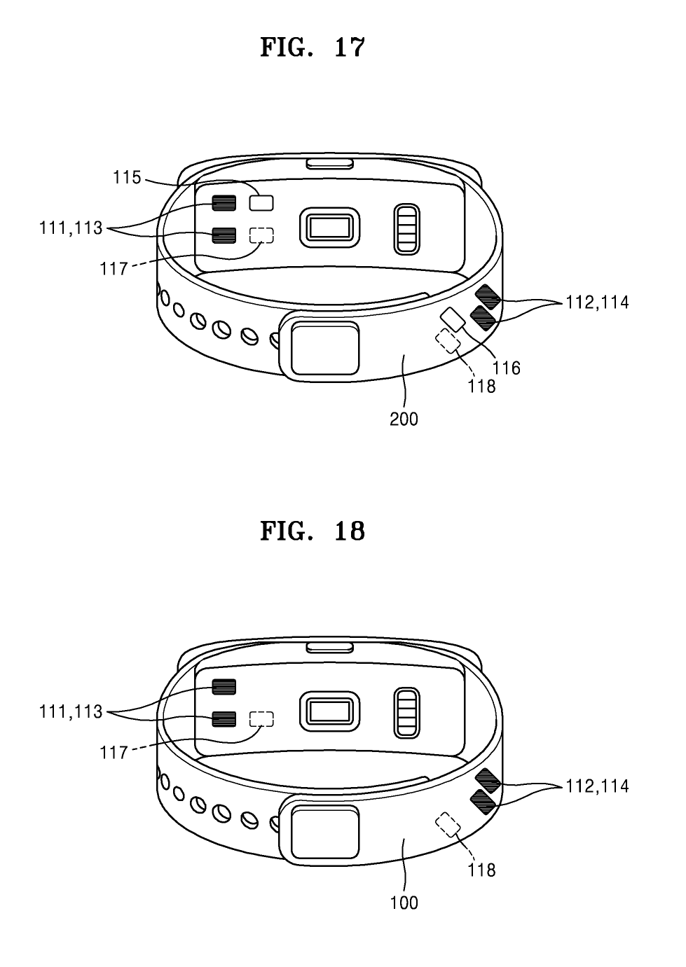

FIG. 17 is a perspective view of an implementation of the body composition measuring apparatus 200 illustrated in FIG. 10. Referring to FIG. 17, the body composition measuring apparatus 200 may be a wearable device, such as a smart watch wearable on a wrist or a smart ring wearable on a finger. The electrode 115 of the 2-point measuring electrode 2P is disposed inside the body composition measuring apparatus 200. Thus, when a user wears the body composition measuring apparatus 200 on his or her wrist, the electrode 115 may be in contact with a skin surface of the wrist. The electrode 116 is disposed outside the body composition measuring apparatus 200. Thus, the user may be able to touch the electrode 116 by his or her finger of the other wrist. The electrodes 111 and 113 of the 4-point measuring electrode 4P are disposed inside the body composition measuring apparatus 200. Thus, when a user wears the body composition measuring apparatus 200 on his or her wrist, the electrodes 111 and 113 may be in contact with a skin surface of the wrist. The electrodes 112 and 114 are disposed outside the body composition measuring apparatus 200. Thus, the user may be able to touch the electrodes 112 and 114 by his or her finger of the other wrist. In a case where the first electrode 110c-1 for measuring the skin hydration is further provided as illustrated in FIGS. 14 and 15, the electrode 117 and the electrode 118 may be respectively disposed inside and outside the body composition measuring apparatus 200.

FIG. 18 is a perspective view of an implementation of the body composition measuring apparatus 200 illustrated in FIG. 11. Referring to FIG. 18, the electrodes 111 and 113 are disposed inside the body composition measuring apparatus 200. Thus, when a user wears the body composition measuring apparatus 200 on his or her wrist, the electrodes 111 and 113 may contact a skin of the wrist. The electrodes 112 and 114 are disposed outside the body composition measuring apparatus 200. Thus, the user is allowed to contact the electrodes 112 and 114 by his or her finger. In a case where the first electrode 110c-1 for measuring the skin hydration is further provided as illustrated in FIG. 16, the electrode 117 and the electrode 118 may be respectively disposed inside and outside the body composition measuring apparatus 200.

In the body composition measuring apparatuses 200 according to the above-described exemplary embodiments, the impedance measuring unit that measures the impedance of the electric circuit including the body may also be used to measure the skin hydration and the body fat ratio. Therefore, it is possible to implement the body composition measuring apparatus 200 capable of measuring the skin hydration and the body fat ratio through a simple configuration.

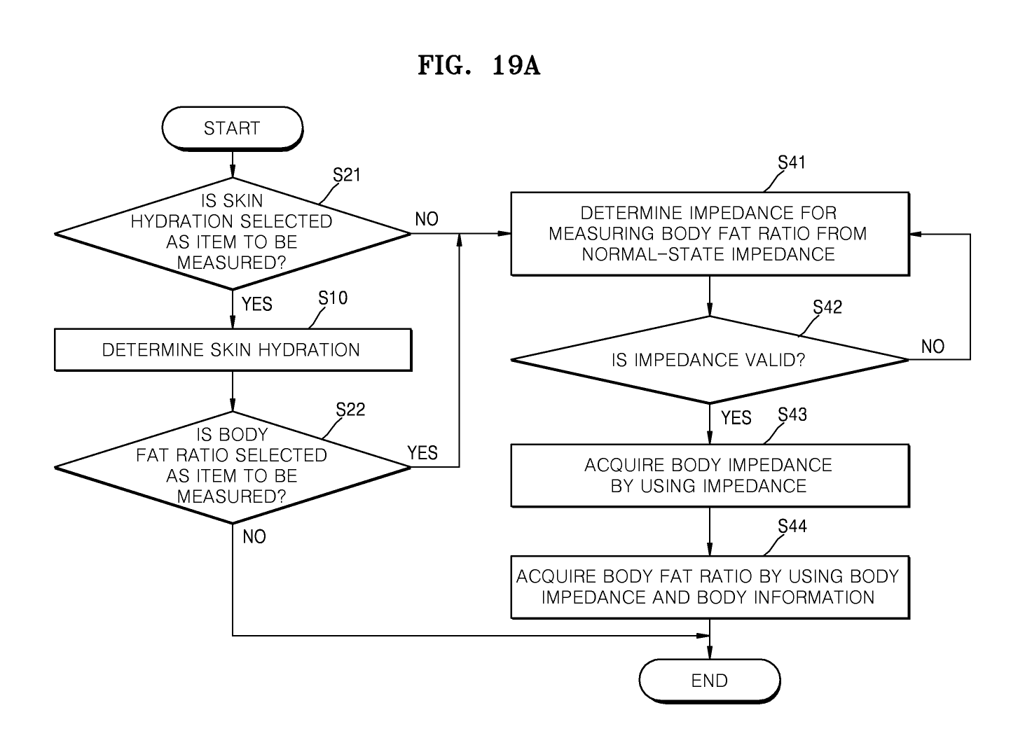

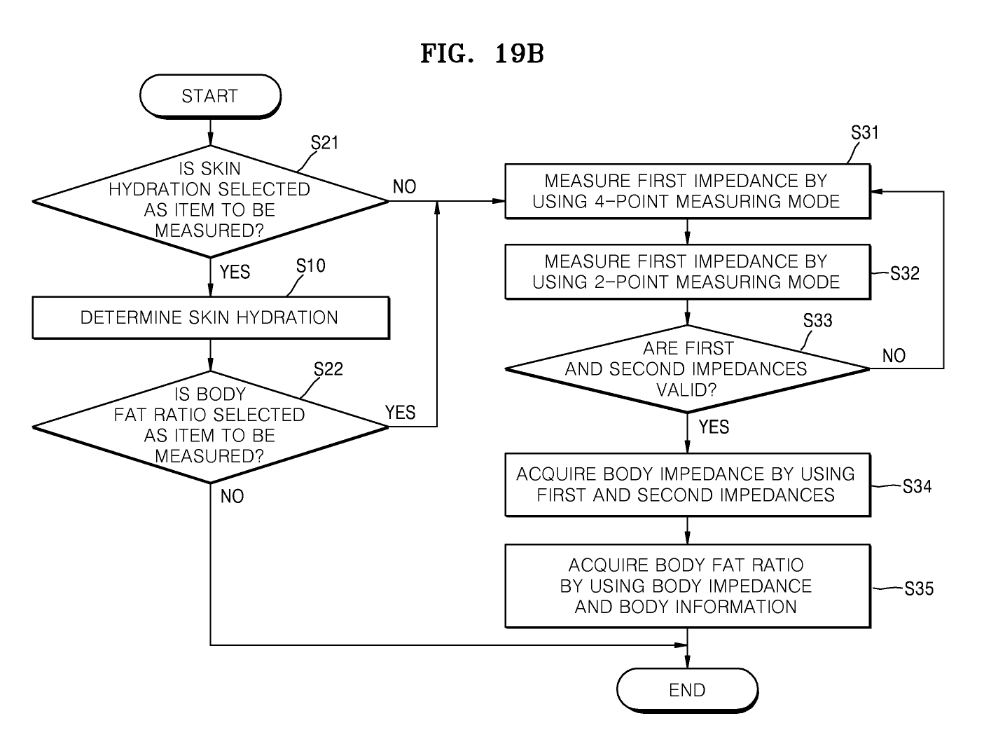

FIGS. 19A and 19B are flowcharts of body composition measuring methods according to exemplary embodiments. Referring to FIGS. 8A, 8B, and 19A, the measurement of the body composition may be started by wearing the body composition measuring apparatus 200 on a wrist and pressing a button or the like.

In operation S21, items to be measured may be determined. The items to be measured may be determined in such a manner that a user wears the body composition measuring apparatus 200 and inputs the items to be measured by pressing the button or the like.

In operation S10, the skin hydration may be determined. Operation S10 may be performed based on the above-described flowchart of FIGS. 8A and 8B.

In operation S11, the impedance of the electric circuit including the body may be measured.

For example, the electrodes 111 and 113 and the electrode 115 may be in contact with the body when the body composition measuring apparatus 200 of FIG. 10 is worn on the wrist of the left hand. According to the impedance measuring mode for measuring the skin hydration, the impedance may be measured by contacting the electrodes 112 and 114 or the electrode 115 with the finger of the right hand. The impedance measuring mode for measuring the skin hydration may be preset in the body composition measuring apparatus 200. The user may select the measuring mode.

For example, the electrodes 111 and 113, the electrode 115, and the electrode 117 may be in contact with the body when the body composition measuring apparatus 200 of FIG. 14 is worn on the wrist of the left hand. The electrode switch 192 may connect the first electrode 110c-1 to the second impedance measuring unit 120a-2. The impedance may be measured when the finger of the right hand comes into contact with the electrode 118.

For example, the electrodes 111 and 113, the electrode 115, and the electrode 117 may be in contact with the body when the body composition measuring apparatus 200 of FIG. 15 is worn on the wrist of the left hand. The electrode switch 192 may connect the first electrode 110c-1 to the first impedance measuring unit 120a-1. The impedance may be measured when the finger of the right hand is in contact with the electrode 118.

For example, the electrodes 111 and 113 may be in contact with the body when the body composition measuring apparatus 200 of FIG. 11 is worn on the wrist of the left hand. According to the impedance measuring mode for measuring the skin hydration, the measuring mode switch 191 may be switched to the 2-point measuring mode or the 4-point measuring mode. The impedance measuring mode for measuring the skin hydration may be preset in the body composition measuring apparatus 200. In addition, the user may select the measuring mode. In this case, the measuring mode switch 191 may be switched to the 2-point measuring mode or the 4-point measuring mode according to the user's selection. The impedance may be measured in the 4-point measuring mode or the 2-point measuring mode when the finger of the right hand comes into contact with the electrodes 112 and 114.

For example, the electrodes 111 and 113 and the electrode 117 may be in contact with the body when the body composition measuring apparatus 200 of FIG. 16 is worn on the wrist of the left hand. The electrode switch 192 may connect the first electrode 110c-1 to the first impedance measuring unit 120a-1. The impedance may be measured when the finger of the right hand comes into contact with the electrode 118.

In operation S12, the waveform detector 130 may determine the normal-state impedance. For example, when the slope of the measurement voltage Vi (or the measurement impedance) with respect to the measurement time is a predetermined value or less, the measurement voltage (Vi) (or the measurement impedance) at that time may be determined as the normal-state voltage (impedance). In addition, the measurement voltage Vi (or the measurement impedance) after a predetermined time that elapses from the start of the measurement may be determined as the normal-state voltage (impedance).

In operation S13, the comparative voltage (impedance) corresponding to n % of the normal-state voltage (impedance) may be determined.

In operation S14, the skin hydration may be determined based on the normal-state voltage (impedance) and the comparative voltage (impedance).

For example, as illustrated in FIG. 8B, in operation S11-2, the measurement voltage Vi (or the measurement impedance obtained by the measurement voltage Vi by the supplied current) and the measurement time may be stored in the memory 131. In addition, in operation S13-2, the time T_{SH} that elapses until the voltage (impedance) changes from the comparative voltage (impedance) to the normal-state voltage (impedance) may be calculated. In operation S14, the skin hydration may be determined from the elapsed time T_{SH} by the preset calculation equation or may be determined by using the look-up table 141.

The determined skin hydration may be provided as visual information to a user through a display.

In operation S21, when only the skin hydration is selected as the item to be measured, the measurement is ended. In operation S21, when only the body fat ratio is selected as the item to be measured, the process proceeds to operation S31 to measure the body fat ratio. In operation S21, when both the skin hydration and the body fat ratio are selected as the item to be measured, the process may proceed from operation S22 to operation S41 after operations S11 to S14 so as to measure the body fat ratio.

In operation S41, the impedance for measuring the body fat ratio may be determined from the normal-state impedance measured in operation S11 of measuring the impedance for measuring the skin hydration. The impedance for measuring the body fat ratio may be an average of impedance values sampled at different points of time in the normal state. In addition, the impedance for measuring the body fat ratio may be an average of normal-state impedance values measured by performing operation S11 twice or more.

In operation S42, the validity of the determined impedance may be determined. During the measurement of the impedance, an impedance measured without contact between the body and the electrode is invalid. Therefore, the body composition measuring apparatus 200 may determine the validity of the measured impedance by checking the state occurring during the measurement of the impedance. As another example, the validity of the impedance may be determined according to the magnitude of the measured impedance. That is, the validity of the impedance may be determined according to whether the measured impedance is within an impedance range that is measurable from the body.

In operation S43, the body determined Zm may be acquired from the determined impedance. For example, the body impedance Zm may be calculated from Equation 1 or 2 above by assuming Zc as “0”. In addition, in a case where average Zc is prestored, the body impedance Zm may be calculated from Equation 1 or 2 by applying the average Zc. In addition, Zm may be acquired from the look-up table that prestores Zm corresponding to the determined impedance.

The body impedance Zm is an impedance necessary for calculating the body fat ratio and is changed according to body characteristics. In operation S44, the body fat ratio acquiring unit 140 may acquire the body fat ratio of the user by using the body impedance Zm and the body information of the user. The body information of the user may be received from the user. The body information of the user may be age, height, or weight of the user.

The determined body fat ratio may be provided as visual information to a user through a display.

An additional exemplary embodiment that acquires the body fat ratio will be described with reference to FIG. 19B.

For example, in the case of the body composition measuring apparatus 200 of FIG. 10, in operation S31, the first impedance measuring unit 120a-1 may supply a constant current to the 4-point measuring electrode 4P and measure the first impedance Z_{4P}. In operation S32, the second impedance measuring unit 120a-2 may supply a constant current to the 2-point measuring electrode 2P and measure the second impedance Z_{2P}.

For example, in the case of the body composition measuring apparatus 200 of FIG. 14, in operation S31, the first impedance measuring unit 120a-1 may supply a constant current to the 4-point measuring electrode 4P and measure the first impedance Z_{4P}. The 2-point measuring electrode 2P may be connected to the second impedance measuring unit 120a-2 by using the electrode switch 192. In operation S32, the second impedance measuring unit 120a-2 may supply a constant current to the 2-point measuring electrode 2P and measure the second impedance Z_{2P}.

For example, in the case of the body composition measuring apparatus 200 of FIG. 15, the 4-point measuring electrode 4P may be connected to the first impedance measuring unit 120a-1 by using the electrode switch 192. In operation S31, the first impedance measuring unit 120a-1 may supply a constant current to the 4-point measuring electrode 4P and measure the first impedance Z_{4P} In operation S32, the second impedance measuring unit 120a-2 may supply a constant current to the 2-point measuring electrode 2P and measure the second impedance Z_{2P}.

For example, in the case of the body composition measuring apparatus 200 of FIG. 11, in operation S31, the measuring mode switch 191 may switch the connection state of the electrode 110b and the impedance measuring unit 120a to the 4-point measuring mode, and the impedance measuring unit 120a may supply a constant current to the electrode 110b and measure the first impedance Z_{4P}. In operation S32, the measuring mode switch 191 may switch the connection state of the electrode 110b and the impedance measuring unit 120a to the 2-point measuring mode, and the impedance measuring unit 120a may supply a constant current to the electrode 110b and measure the second impedance Z_{2P}.

For example, in the case of the body composition measuring apparatus 200 of FIG. 16, the electrode switch 192 may connect the second electrode 110c-2 to the impedance measuring unit 120a. In operation S31, the measuring mode switch 191 may switch the connection state of the second electrode 110c-2 and the impedance measuring unit 120a to the 4-point measuring mode, and the impedance measuring unit 120a may supply a constant current to the second electrode 110c-2 and measure the first impedance Z_{4P}. In operation S32, the measuring mode switch 191 may switch the connection state of the second electrode 110c-2 and the impedance measuring unit 120a to the 2-point measuring mode, and the impedance measuring unit 120a may supply a constant current to the second electrode 110c-2 and measure the second impedance Z_{2P}.

Operations S31 and S32 may be performed twice or more, and the first and second impedances Z_{4P} and Z_{2P} may be an average of values obtained from measuring twice or more. Each of operations S31 and S32 may be performed once, and the first and second impedances Z_{4P} and Z_{2P} may be an average of measured values sampled at different points of time in the normal state.

In operation S33, the body composition measuring apparatus 200 may determine the validity of the firs impedance Z_{4P} and the second impedance Z_{2P} For example, the body composition measuring apparatus 200 may determine the validity of the firs impedance Z_{4P} and the second impedance Z_{2P} according to the contact state. During the measurement of the impedance, an impedance measured without contact between the body and the electrode is invalid. Therefore, the body composition measuring apparatus 200 may determine the validity of the measured impedance by checking the state occurring during the measurement of the impedance. As another example, the validity of the impedance may be determined according to the magnitude of the measured impedance. That is, the validity of the impedance may be determined according to whether the measured impedance is within an impedance range that is measurable from the body.

In operation S34, the body impedance acquiring unit 150 may calculate the body impedance Zm by using the first and second impedances Z_{4P} and Z_{2P} and the first and second relational expressions. Alternatively, the body impedance acquiring unit 150 may acquire the body impedance Zm from the look-up table 153 by using the first and second impedances Z_{4P} and Z_{2P}.

In operation S35, the body fat ratio acquiring unit 160 may acquire the body fat ratio of the user by using the body impedance Zm and the body information of the user. For example, the body fat ratio may be determined based on the body impedance and the input user information such as the age, height, and weight of the user.

The determined body fat ratio may be provided as visual information to the user through a display.

The apparatuses according to the exemplary embodiments may include a processor, a memory storing and executing program data, a permanent storage such as a disk drive, a communication port communicating with an external device, and a user interface such as a touch panel, a key, or a button.

In addition, other exemplary embodiments can also be implemented through computer-readable code/instructions in/on a medium, e.g., a computer-readable medium, to control at least one processing element to implement any above-described exemplary embodiment. The medium can correspond to any medium/media permitting the storage and/or transmission of the computer-readable code.

The computer-readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as Internet transmission media. Thus, the medium may be such a defined and measurable structure including or carrying a signal or information, such as a device carrying a bitstream according to one or more exemplary embodiments. The media may also be a distributed network, so that the computer-readable code is stored/transferred and executed in a distributed fashion. Furthermore, the processing element could include a processor or a computer processor, and processing elements may be distributed and/or included in a single device.

Disclosed exemplary embodiments may be described in terms of functional block components and various processing steps. Such functional blocks may be realized by any number of hardware and/or software components configured to perform the specified functions. For example, the exemplary embodiments may employ various integrated circuit components (e.g., memory elements, processing elements, logic elements, look-up tables, and the like) that may carry out a variety of functions under the control of one or more processors or other control devices. Similarly, where the elements of the exemplary embodiments are implemented using software programming or software elements, the exemplary embodiments may be implemented with any programming or scripting language such as C, C++, Java, assembler, or the like, using any combination of data structures, objects, processes, routines, and other programming elements. Functional aspects may be implemented as instructions executed by one or more processors. Furthermore, the exemplary embodiments could employ any number of conventional techniques for electronics configuration, signal processing, control, data processing, and the like. The words “mechanism” and “element” are used broadly and are not limited to mechanical or physical embodiments, but can include software routines in conjunction with processors, etc.

The particular implementations shown and described herein are illustrative examples and are not intended to otherwise limit the scope of this disclosure in any way. For the sake of brevity, conventional electronics, control systems, software development, and other functional aspects of the systems may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the exemplary embodiments unless the element is specifically described as “essential” or “critical”.

The use of the terms “a,” “an,” “the,” and similar referents in the context of describing the exemplary embodiments (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. The steps of all methods described herein can be performed in any suitable order unless otherwise indicated herein or otherwise clearly contradicted by context. Moreover, one or more of the blocks and/or interactions described may be changed, eliminated, sub-divided, or combined; and disclosed processes may be carried out sequentially and/or carried out in parallel by, for example, separate processing threads, processors, devices, discrete logic, circuits, etc. The examples provided herein and the exemplary language (e.g., “such as” or “for example”) used herein are intended merely to better illuminate the exemplary embodiments and does not pose a limitation on the scope of this disclosure unless otherwise claimed. In view of this disclosure, numerous modifications and adaptations will be readily apparent to those skilled in this art without departing from the spirit and scope of this disclosure.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A body composition measuring apparatus comprising:
an electrode configured to be in contact with a body of a user;
an impedance measuring unit connected to the electrode and configured to measure an impedance of the body via the electrode; and
a first determining unit configured to acquire skin hydration based on a normal-state impedance of the body and a comparative impedance corresponding to a percentage of the normal-state impedance.

2. The body composition measuring apparatus of claim 1, wherein the first determining unit comprises:
a waveform detector configured to determine the normal-state impedance and the comparative impedance; and
a skin hydration acquiring unit configured to determine the skin hydration based on the normal-state impedance and the comparative impedance.

3. The body composition measuring apparatus of claim 2, wherein
the waveform detector is further configured to determine a difference between a measurement time corresponding to the normal-state impedance and a measurement time corresponding to the comparative impedance, and
the skin hydration acquiring unit is further configured to determine the skin hydration based on the determined difference.

4. The body composition measuring apparatus of claim 1, wherein the percentage is about 95% to about 98%.

5. The body composition measuring apparatus of claim 1, further comprising a second determining unit configured to acquire a body fat ratio based on the measured impedance of the body.

6. The body composition measuring apparatus of claim 5, wherein the electrode comprises a first electrode configured to measure the skin hydration, and a second electrode configured to measure the body fat ratio, and
the body composition measuring apparatus further comprises an electrode switch configured to selectively connect the first and second electrodes to the impedance measuring unit.

7. The body composition measuring apparatus of claim 5, wherein the impedance measuring unit is further configured to measure a first impedance in a 4-point measuring mode and measure a second impedance in a 2-point measuring mode, and
the second determining unit comprises:
a body impedance acquiring unit configured to determine the body impedance of the user based on the first impedance and the second impedance; and
a body fat ratio acquiring unit configured to acquire the body fat ratio of the user based on the body impedance and body information of the user.

8. The body composition measuring apparatus of claim 7, wherein the electrode further comprises a measuring mode switch configured to selectively connect the electrode to the impedance measuring unit in the 4-point measuring mode and the 2-point measuring mode.

9. The body composition measuring apparatus of claim 7, wherein the electrode comprises a 4-point measuring electrode for 4-point measurement and a 2-point measuring electrode for 2-point measurement, and
the impedance measuring unit comprises:
a first impedance measuring unit connected to the 4-point measuring electrode to measure the first impedance; and
a second impedance measuring unit connected to the 2-point measuring electrode to measure the second impedance.

10. The body composition measuring apparatus of claim 9, wherein
the electrode further comprises a first electrode configured to measure the skin hydration, and
the body composition measuring apparatus further comprises an electrode switch configured to selectively connect the first electrode to one of the first and second impedance measuring units.

11. A body composition measuring method comprising:
measuring an impedance of a body of a user;
determining a normal-state impedance of the body;
determining a comparative impedance corresponding to a percentage of the normal-state impedance; and
determining skin hydration based on the normal-state impedance and the comparative impedance.

12. The body composition measuring method of claim 11, wherein the determining the skin hydration comprises:
determining a difference of a measurement time corresponding to the normal-state impedance and a measurement time corresponding to the comparative impedance; and
determining the skin hydration based on the determined difference.

13. The body composition measuring method of claim 11, wherein the percentage is about 95% to about 98%.

14. The body composition measuring method of claim 11, further comprising acquiring a body fat ratio based on the measured impedance.

15. The body composition measuring method of claim 14, wherein the body fat ratio is acquired based on the normal-state impedance.

16. The body composition measuring method of claim 14, wherein the measuring the impedance comprises selectively connecting an impedance measuring unit to a first electrode configured to measure the skin hydration and a second electrode configured to measure a body fat ratio.

17. The body composition measuring method of claim 14, wherein the measuring the impedance comprises:
measuring a first impedance in a 4-point measuring mode; and
measuring a second impedance in a 2-point measuring mode, and
the acquiring the body fat ratio comprises:
determining the body impedance based on the first impedance and the second impedance; and
acquiring the body fat ratio of the user based on the body impedance and body information of the user.

18. The body composition measuring method of claim 17, wherein the measuring the impedance comprises selectively switching a connection mode of an electrode and an impedance measuring unit to the 4-point measuring mode and the 2-point measuring mode.
